(19) United States
(12) Patent Application Publication
(10) Pub. No.: US 2025/0064600 A1
(43) Pub. Date: Feb. 27, 2025

(54) SYSTEM AND INSTRUMENTS FOR DELIVERING BONE GRAFT MATERIAL

(71) Applicant: Solmy Moran, West New York, NJ (US)

(72) Inventor: Solmy Moran, West New York, NJ (US)

(21) Appl. No.: 18/812,642

(22) Filed: Aug. 22, 2024

Related U.S. Application Data
(60) Provisional application No. 63/534,250, filed on Aug. 23, 2023.

(51) Int. Cl. A61F 2/46; A61F 2/28

(52) U.S. Cl. CPC A61F 2/4601; A61F 2/4611; A61F 2002/2835

(57) ABSTRACT

A system and instruments for delivery of bone graft material to a patient during an open surgery, such as in spinal fusion surgeries, in a simple, convenient and time-efficient manner are provided. The system comprises a collection device for collecting milled tissue from the patient, and a delivery instrument for delivering the autograft tissue to the surgical site.

### CROSS-REFERENCE TO RELATED APPLICATIONS

This application claims the benefit of U.S. Provisional No. 63/534,250 filed on Aug. 23, 2023 and entitled “Bone Graft Inserter,” the contents of which are incorporated in their entirety by reference.

### FIELD

The system and associated devices described herein generally relate to medical instruments for use in medical procedures such as surgeries. More particularly, a medical kit comprising instruments for the delivery of bone graft material to a patient, such as during an open surgical procedure, is provided.

### BACKGROUND

Bone grafting is a surgical procedure that uses transplanted bone or similar substances to repair and rebuild diseased or damaged bone. This surgical procedure can promote the patient's natural bone-making process by reinforcing gaps in weak, broken or deficient bones with the graft material to provide mechanical support. Bone grafting can also stimulate tissue growth to bridge gaps in adjacent bone segments and restore bone strength and rigidity. The procedure may be necessary if bones don't heal correctly after a fracture. For example, bone grafting may be required where there is a delayed union and the bone healing, or bone fusion, occurs more slowly that desired, or where there is a malunion, where the bones heal in an abnormal position, or where there is a nonunion, where there is no bone fusion.

Some of the reasons for bone grafting may include treatment of bone diseases like osteonecrosis, bone cancer, or osteomylelitis (bone infection), or for dental bone reinforcement, joint replacement or repair surgery, spinal fusion, or trauma such as bad fractures or shattered bones. In these examples, the bone grafting material is used to promote bone fusion and the patient's natural healing.

The material for the bone grafting can come from the patient (i.e., autograft) or from a donor (i.e., allograft). Other methods of bone grafting can use bone marrow aspirate or synthetic bone graft materials. These synthetic bone graft materials can include calcium phosphate ceramics, bioactive glasses, polymers, or even metals, and can be combined with growth factors.

Although allograft material is commonly used in spinal fusion surgeries, a procedure where two or more spinal bones are fused together to form a rigid, non-moving spinal segment, autograft material is preferable over allograft material. Using autograft material provides the benefit of using the patient's own tissue which increases the chances of successful fusion by decreasing the risk of foreign tissue rejection. The autograft tissue typically comes from the top of the patient's hip bone (iliac crest), but can also be the bone tissue that is removed or displaced from the insertion of spinal implants (i.e., bone screws, fusion cages, vertebral body replacements, and the like) into the spinal bone tissue.

Currently, a typical method for applying autograft material in spinal fusion surgeries involves collecting the harvested patient bone tissue (whether obtained from the iliac crest or the displaced spinal bone tissue), processing the collected bone tissue by grinding the tissue to a desired size or shape, and then re-introducing the milled pieces of this harvested bone tissue back to the surgical site. In many cases, the target area (which can be, for example, the intervertebral space, the posterior lateral spinal region, or even the top of the pedicle bone screws) is packed with the milled pieces of the harvested bone tissue to provide a framework around which healthy bone tissue can grow. The packing is currently done by hand or with forceps, resulting in a cumbersome and time-consuming process that prolongs the operating room (“OR”) time for both the surgical staff and the patient, not to mention the time the patient has to be under anesthesia.

It is therefore desirable to provide a system that facilitates accurate delivery of autograft material to a patient during an open surgery, such as in spinal fusion surgeries, in a simple, convenient and time-efficient manner.

### SUMMARY

The following presents a simplified summary in order to provide a basic understanding of some aspects of the present system and instruments. This summary is not an extensive overview of the claimed subject matter. It is intended to neither identify key or critical elements of the claimed subject matter nor delineate the scope of the claimed subject matter. Its sole purpose is to present some concepts of the present system and instruments in a simplified form as a prelude to the more detailed description that is presented later.

In accordance with one aspect of the disclosure, a system for delivering bone graft material is provided. The system can comprise a delivery instrument having a barrel with a main tubular body extending between a first open end and a second open end. The system can further comprise an end cap configured for releasable attachment to the second open end of the barrel. The end cap can have a plurality of through holes. The system can also comprise a plunger for insertion into the main tubular body. A collection device may also be provided with the system. The collection device can be configured as a generally conically shaped funnel and having a first open end and a second open end, wherein the second open end is configured for attachment to the first open end of the body of the delivery instrument.

In some embodiments, the barrel can include gripping handles. The barrel can also include indicia on an outer surface of the main tubular body.

In some embodiments, the second open end of the main tubular body can have external threads and the end cap can include internal threads for a threaded engagement with the second open end of the main tubular body.

In some embodiments, the first open end of the barrel can be configured to releasably attach to the second open end of the collection device.

In some embodiments, the plunger can include an enlarged head portion at a first end. The plunger can also include a conformable flange near a second, opposed end.

The system may be formed of a medical grade plastic.

In some embodiments, the collection device may be configured for attachment to a bone mill.

In accordance with another aspect of the disclosure, a bone graft delivery instrument is provided. The bone graft delivery instrument may comprise a barrel with a main tubular body extending between a first open end and a second open end. The instrument can further comprise an end cap configured for releasable attachment to the second open end of the barrel. The end cap can have a plurality of through holes. The instrument can also comprise a plunger for insertion into the main tubular body. The plunger can have a head portion at a first end for depressing the elongate plunger, and a contact surface at an opposed, second end.

In some embodiments, the barrel can include gripping handles. The barrel can also include indicia on an outer surface of the main tubular body. The barrel may also be transparent or opaque.

In some embodiments, the second open end of the main tubular body can have outer threads and the end cap can include internal threads for a threaded engagement with the second open end of the main tubular body.

In some embodiments, the first open end of the barrel can be configured to releasably attach to a collection device for receiving milled bone tissue.

In some embodiments, the plunger can include an enlarged head portion at the first end. The plunger can also include a conformable flange near the opposed, second end. In some embodiments, the conformable flange can comprise a silicone, rubber or other suitable polymeric ring. In some embodiments, the plunger can include a protrusion near the first end to prevent overextension of the plunger into the main tubular body.

The delivery instrument may be formed of a medical grade plastic.

It is to be understood that both the foregoing general description and the following detailed description are exemplary and explanatory only and are not restrictive of the disclosure. Additional features of the present system and instruments will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the present system and instruments, and together with the description serve to explain the principles of the disclosure.

FIG. 1 shows an exemplary system or instrument kit of the present disclosure.

FIG. 2 shows a perspective view of an exemplary barrel of the delivery instrument of the instrument kit of FIG. 1.

FIG. 3A shows a top-down view of an exemplary end cap of the instrument kit of FIG. 1.

FIG. 3B shows a bottom-up view of an exemplary end cap of the instrument kit of FIG. 3A.

FIG. 4 shows an exploded view of an exemplary delivery instrument of the instrument kit of FIG. 1.

FIGS. 5A to 5C show a method of using the instruments of the instrument kit of FIG. 1, in which:

FIG. 5A illustrates the step of collecting and filling the barrel of the delivery instrument of FIG. 1 with bone graft material;

FIG. 5B illustrates the step of assembling the delivery instrument of FIG. 5A; and

FIG. 5C is a partial cutaway view of the delivery instrument and illustrates the step of delivering the bone graft material using the delivery instrument of FIG. 5B.

### DETAILED DESCRIPTION

A system for delivering bone graft material in an accurate, simple-to-use, low cost and expedient manner is described herein. The system may comprise a medical or surgical kit that includes various instruments for collecting and delivering the bone graft material, which may be autograft material, for use in an open surgical procedure such as, for example, orthopedic surgeries such as a spinal fusion procedure.

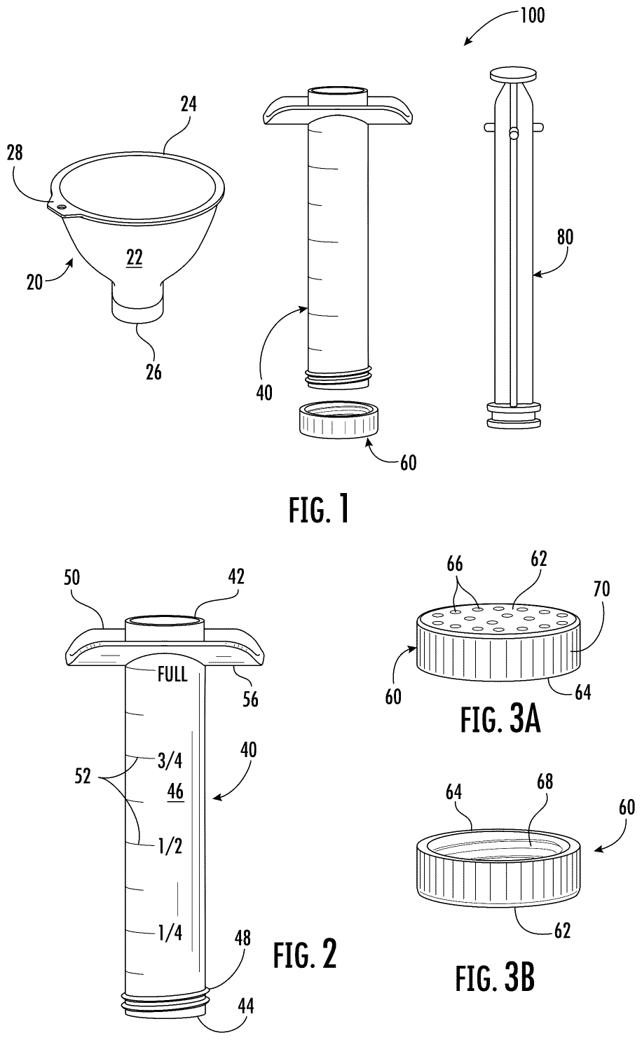

An exemplary system 100 for collecting and delivering bone graft material is illustrated in FIG. 1. The system 100 may comprise a collection device 20, shown here configured as a funnel, and a delivery instrument 30 that comprises a tubular body or barrel 40, an end cap 60, and a plunger 80. The collection device 20 may have a main body 22 having a generally conical shape to act as a funnel, a first open end 24 and an opposed, second end 26. A tab 28 may be provided for case of handling or gripping the collection device 20. The first open end 24 of the collection device 20 may be sized and shaped for releasable attachment to a bone mill 120, a machine often found in surgical operating rooms for orthopedic procedures to grind up a patient's bone to be placed back into the same patient. The second open end 26 of the collection device 20 may be sized and shaped to attach to a barrel 40 of the delivery instrument 30, which barrel 40 receives the milled or ground patient bone tissue for use as bone grafting material, as will be described in greater detail below.

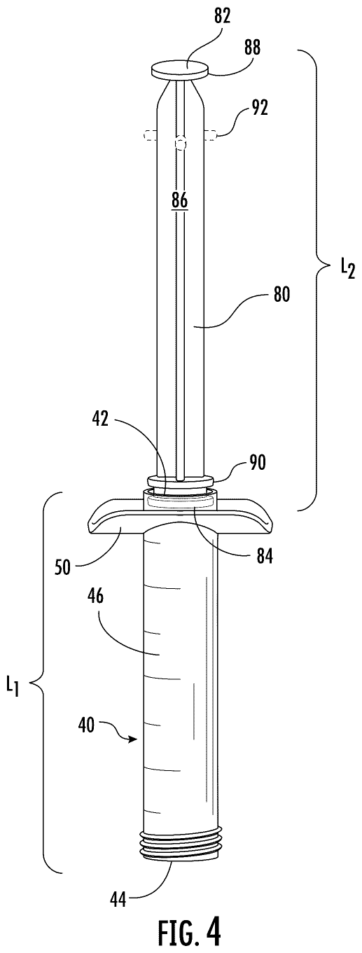

FIG. 2 shows the barrel 40 of the delivery instrument 30 in greater detail. The barrel 40 may have an elongate, tubular body 46 with a first open end 42 and an opposed, second open end 44. The first open end 42 may be configured and sized to connect to the second open end 26 of the collection device 20. The connection may be a threaded connection, friction fit connection, dovetail connection, or similar for convenient and secure attachment and release. The second open end 44 of the barrel 40 may include external threads 48 for receiving an end cap 60. Gripping handles 50 may be provided near the first open end 42 of the barrel 40. As shown, the gripping handles 50 may be contoured with a curved surface 56 to provide the user with a comfortable grip. The elongate, tubular body 46 may include indicia 52 for use as a fill line and in identifying the amount of bone graft material contained inside the barrel 40. The scale of the indicia 52 may be in any suitable unit, such as for example, in cc or mL, or as a ratio or percentage filled, such as ¼ or ½ full, or 25% or 100% full, with any number of increments as desired. In an exemplary embodiment, the barrel 40 may be formed of a transparent or opaque material so that the user is able to see how much content is inside.

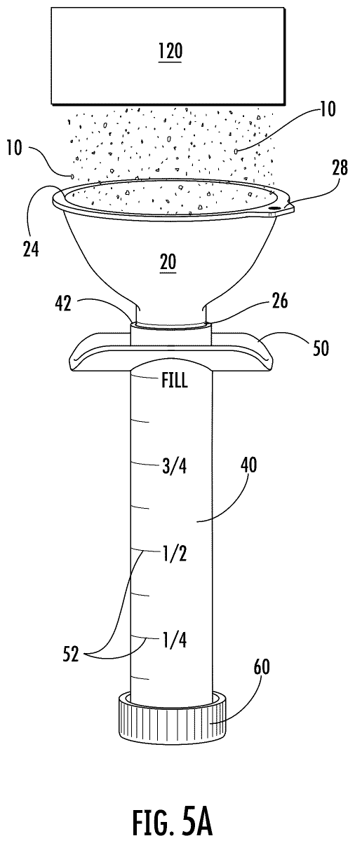

FIGS. 3A and 3B illustrate details of the detachable end cap 60. As shown in FIG. 3A, the end cap 60 includes an outer, exterior surface 62 from which one or more through holes 66 extend. These through holes can serve as air vents, and may range in size from about 14 to 18 gauge needle tip, as an example. Of course, the holes may be any size or shape as desired to allow air to be released while preventing the graft material 10 inside the barrel 40 to escape. As shown in FIG. 3B, the lower, interior surface 64 of the end cap 60 can include internal threads 68 to allow the end cap 60 to threadedly mate with the outer threads 48 at the second open end 44 of the barrel 40. The exterior surface of the end cap 60 may have ridges or surface protrusions 70, as shown, to enhance handling and case of gripping.

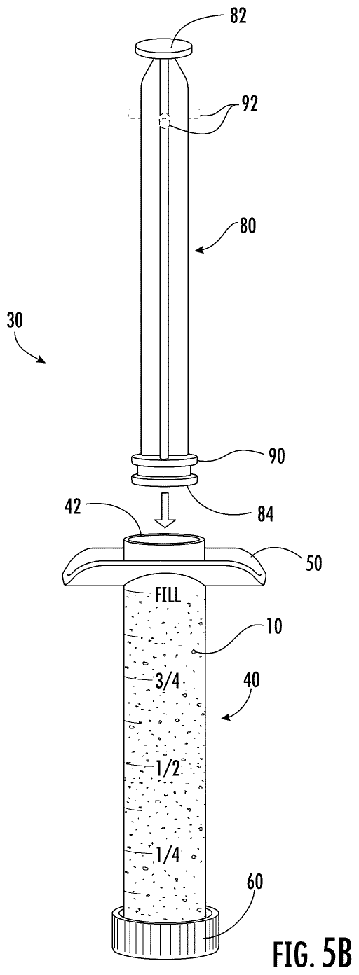

FIG. 4 shows an exploded view of the delivery instrument 30, in which a plunger 80 may be provided for pushing the contents of the barrel 40 out the second open end 44. The plunger 80 may comprise an elongate shaft 86 extending between a first end 82 and a second, opposed end 84. The first end 82 may have an enlarged head 88 configured to facilitate pushing with the user's thumb. In some embodiments, the diameter of the enlarged head 88 may be wider than the diameter of the first open end 42 of the barrel 40 so that the enlarged head 88 does not extend into the barrel 40. In an exemplary embodiment, the enlarged head 88 may have a depression (not shown) for the thumb to easily rest against the outer surface of the enlarged head 88. As shown, the elongate shaft 86 may be shaped like an “X” or “+” in cross-section. However, it is understood that other cross-sectional shapes can be used and envisioned. The second, opposed end 84 of the plunger 80 may have a flat outer contact surface and have near it a conformable flange 90 that allows the plunger 80 to have a tight fit within the barrel 40, while still allowing sliding movement inside the barrel 40 during depression. The conformable flange 90 may be a silicone or rubber ring, for example. Of course, other suitable polymeric materials may also be used for the flange 90. If desired, additional flanges or protrusions 92 may be provided around the plunger 80, as shown, near the enlarged head 88 to prevent the plunger 80 from pushing too far into the barrel 40. The optional protrusions 92 can act as a stopper when they contact the first open end 42, keeping the plunger 80 from further insertion into the barrel 40.

It is understood that the barrel 40 may have a length L₁ while plunger 80 may have a length L₂ that is greater than L₁. In one embodiment, the barrel 40 may have a length L₁ of about 4 to 6 inches, while the plunger 80 may have a length L₂ of about 5 to about 7 inches, for example. Of course, it is understood that any size dimensions are merely for illustrative purposes and that the instruments can have any suitable size dimension that is convenient for use with already existing medical equipment, devices and instruments commonly found in surgical operating rooms.

Similarly, it is well understood that the instruments 20, 40, 60, 80 of the medical kit 100 may be formed of any medical grade material such as a medical grade plastic. The instruments 20, 40, 60, 80 may be disposable, or reusable after sterilization.

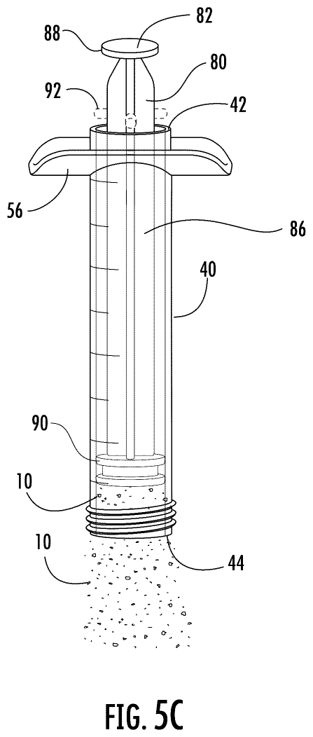

FIGS. 5A to 5C show an exemplary method of using the medical kit 100.

### Filling

To fill the delivery instrument 30, the collection device 20 may be attached to, or placed under a bone mill 120, as shown in FIG. 5A. Bone tissue can be inserted into the bone mill 120 for processing into a suitable size and shape for the bone graft material 10. As described above, the bone tissue can be the patient's own bone tissue, either harvested from the iliac crest or other body part, or the bone tissue is displaced bone tissue that has been removed during the surgery. It is understood, of course, that additional or alternative materials may also be introduced into the bone mill 120 to be incorporated into the final bone graft material 10, including for example, bone marrow aspirate, growth factors or other bioactive agents. The bone mill 120 is a machine that is typically already present in a surgical operating room, particularly for orthopedic surgeries. The medical kit 100 of the present disclosure is therefore a convenient accessory for fusion surgeries and the like. After the end cap 60 is attached to the barrel 40, as shown, the barrel 40 can be attached to the collection device 10 to receive the milled bone tissue 10.

### Assembling

When the desired amount of bone graft material 10 has filled the barrel 40, the collection device 20 can be detached from, or moved away from (in the event they are not attached together), the bone mill 120. The filled barrel 40 can be detached from, or moved away from (in the event they are not attached together), the collection device 20. Next, the plunger 80 may be inserted into the barrel 40, as shown in FIG. 5B. The through holes 66 of the attached end cap 60 can serve as air vents and enable air to escape, allowing the plunger 80 to be inserted into the barrel 40 and compact the graft material 10 with its flat outer contact surface at the second end 84 towards the second open end 44 of the barrel without great resistance. The conformable flange or ring 90 at the second end 84 can provide a counterforce to prevent the plunger 80 from traveling too quickly through the barrel 40.

### Dispensing

Once the delivery instrument 30 has been assembled, the bone graft material 10 can now be dispensed. To dispense the bone graft material 10, the end cap 60 is removed, as shown in FIG. 5C. While grabbing the gripping handles 50 with fingers, the user can depress the plunger 80 with the thumb to apply force from the plunger 80 on to the bone graft material 10 and move it out of the second open end 44 of the barrel 40 and to the target surgical site.

It is contemplated that one or more of the delivery instruments 30 may be prepared by repeating the above steps and be quickly filled with bone graft material 10. The plurality of prepared delivery instruments 30 can be available at the same time as the open surgical procedure is being performed. The prepared delivery instruments 30 would then be ready for use by the operating surgeon or staff easily and conveniently, and be dispensed either in stages or all at once, without requiring additional prep time at the end of the surgery and thus reducing the overall surgery time and the time the patient is under anesthesia.

It should be understood that the medical kit 100 and its instruments 20, 40, 60, 80 are described for use in a surgical fusion procedure, this system can be used for any open surgical procedure that can benefit from, or require, a bone graft, such as for treatment of bone diseases like osteonecrosis, bone cancer, or osteomylelitis (bone infection), or for dental bone reinforcement, or joint replacement or repair surgery.

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

What we claim is:

1. A system for delivering bone graft material, comprising:
a delivery instrument comprising a barrel having a main tubular body extending between a first open end and a second open end, an end cap configured for releasable attachment to the second open end of the barrel, the end cap having a plurality of through holes, and a plunger for insertion into the main tubular body; and
a collection device configured as a generally conically shaped funnel and having a first open end and a second open end, wherein the second open end is configured for attachment to the first open end of the body of the delivery instrument.

2. The system of claim 1, wherein the barrel includes gripping handles.

3. The system of claim 1, wherein the barrel includes indicia on an outer surface of the main tubular body.

4. The system of claim 1, wherein the second open end of the main tubular body has outer threads and the end cap includes internal threads for a threaded engagement with the second open end of the main tubular body.

5. The system of claim 1, wherein the first open end of the barrel is configured to releasably attach to the second open end of the collection device.

6. The system of claim 1, wherein the plunger includes an enlarged head portion at a first end.

7. The system of claim 1, wherein the plunger includes a conformable flange near a second, opposed end.

8. The system of claim 1, wherein the delivery instrument is formed of a medical grade plastic.

9. The system of claim 1, wherein the collection device is configured for attachment to a bone mill.

10. A bone graft delivery instrument, comprising:
a barrel having a main tubular body extending between a first open end and a second open end;
an end cap configured for releasable attachment to the second open end of the main tubular body, the end cap having a plurality of through holes; and
an elongate plunger for insertion into the main tubular body, the plunger having a head portion at a first end for depressing the elongate plunger and a contact surface at an opposed, second end.

11. The instrument of claim 10, wherein the barrel includes gripping handles.

12. The instrument of claim 10, wherein the barrel includes indicia on an outer surface of the main tubular body.

13. The instrument of claim 10, wherein the second open end of the main tubular body has outer threads and the end cap includes internal threads for a threaded engagement with the second open end of the main tubular body.

14. The instrument of claim 10, wherein the first open end of the barrel is configured to releasably attach to a collection device for receiving milled bone tissue.

15. The instrument of claim 10, wherein the plunger includes an enlarged head portion at the first end.

16. The instrument of claim 10, wherein the plunger includes a conformable flange near the opposed, second end.

17. The instrument of claim 15, wherein the conformable flange comprises a silicone or rubber ring.

18. The instrument of claim 10, wherein the plunger includes a protrusion near the first end to prevent overextension of the plunger into the main tubular body.

19. The instrument of claim 10, wherein the delivery instrument is formed of a medical grade plastic.

20. The instrument of claim 10, wherein the barrel is transparent or opaque.
